SEQUENCE LISTING (67 sequences)

SEQ ID NO: 1 - RNA, 41 nt, Artificial Sequence
  Synthetic Nucleotide
uaauuucuacuaaguguagaugggguuugagguccauuaca

SEQ ID NO: 2 - DNA, 25 bp, Artificial Sequence
  Synthetic Nucleotide
accccatctacacttagtagaaatt

SEQ ID NO: 3 - DNA, 24 bp, Artificial Sequence
  Synthetic Nucleotide
accccatctacacttagtagaaat

SEQ ID NO: 4 - DNA, 23 bp, Artificial Sequence
  Synthetic Nucleotide
accccatctacacttagtagaaa

SEQ ID NO: 5 - DNA, 22 bp, Artificial Sequence
  Synthetic Nucleotide
accccatctacacttagtagaa

SEQ ID NO: 6 - DNA, 21 bp, Artificial Sequence
  Synthetic Nucleotide
accccatctacacttagtaga

SEQ ID NO: 7 - DNA, 19 bp, Artificial Sequence
  Synthetic Nucleotide
accccatctacacttagta

SEQ ID NO: 8 - DNA, 18 bp, Artificial Sequence
  Synthetic Nucleotide
accccatctacacttagt

SEQ ID NO: 9 - DNA, 17 bp, Artificial Sequence
  Synthetic Nucleotide
accccatctacacttag

SEQ ID NO: 10 - DNA, 16 bp, Artificial Sequence
  Synthetic Nucleotide
accccatctacactta

SEQ ID NO: 11 - DNA, 15 bp, Artificial Sequence
  Synthetic Nucleotide
accccatctacactt

SEQ ID NO: 12 - DNA, 25 bp, Artificial Sequence
  Synthetic Nucleotide
ctcaaaccccatctacacttagtag

SEQ ID NO: 13 - DNA, 24 bp, Artificial Sequence
  Synthetic Nucleotide
tcaaaccccatctacacttagtag

SEQ ID NO: 14 - DNA, 23 bp, Artificial Sequence
  Synthetic Nucleotide
caaaccccatctacacttagtag

SEQ ID NO: 15 - DNA, 22 bp, Artificial Sequence
  Synthetic Nucleotide
aaaccccatctacacttagtag

SEQ ID NO: 16 - DNA, 21 bp, Artificial Sequence
  Synthetic Nucleotide
aaccccatctacacttagtag

SEQ ID NO: 17 - DNA, 20 bp, Artificial Sequence
  Synthetic Nucleotide
accccatctacacttagtag

SEQ ID NO: 18 - DNA, 19 bp, Artificial Sequence
  Synthetic Nucleotide
ccccatctacacttagtag

SEQ ID NO: 19 - DNA, 18 bp, Artificial Sequence
  Synthetic Nucleotide
cccatctacacttagtag

SEQ ID NO: 20 - DNA, 17 bp, Artificial Sequence
  Synthetic Nucleotide
ccatctacacttagtag

SEQ ID NO: 21 - DNA, 16 bp, Artificial Sequence
  Synthetic Nucleotide
catctacacttagtag

SEQ ID NO: 22 - DNA, 15 bp, Artificial Sequence
  Synthetic Nucleotide
atctacacttagtag

SEQ ID NO: 23 - DNA, 21 bp, Artificial Sequence
  Synthetic Nucleotide
atctacacttagtagaaatta

SEQ ID NO: 24 - DNA, 17 bp, Artificial Sequence
  Synthetic Nucleotide
tgtaatggacctcaaac

SEQ ID NO: 25 - DNA, 16 bp, Artificial Sequence
  Synthetic Nucleotide
tgtaatggacctcaaa

SEQ ID NO: 26 - DNA, 15 bp, Artificial Sequence
  Synthetic Nucleotide
tgtaatggacctcaa

SEQ ID NO: 27 - DNA, 14 bp, Artificial Sequence
  Synthetic Nucleotide
tgtaatggacctca

SEQ ID NO: 28 - DNA, 13 bp, Artificial Sequence
  Synthetic Nucleotide
tgtaatggacctc

SEQ ID NO: 29 - DNA, 12 bp, Artificial Sequence
  Synthetic Nucleotide
tgtaatggacct

SEQ ID NO: 30 - DNA, 11 bp, Artificial Sequence
  Synthetic Nucleotide
tgtaatggacc

SEQ ID NO: 31 - DNA, 59 bp, Artificial Sequence
  Synthetic Nucleotide
ctgatagtatttaggggtttgaggtccattacagctgtaatgaacattacgtcttatgt

SEQ ID NO: 32 - DNA, 59 bp, Artificial Sequence
  Synthetic Nucleotide
acataagacgtaatgttcattacagctgtaatggacctcaaacccctaaatactatcag

SEQ ID NO: 33 - RNA, 20 nt, Artificial Sequence
  Synthetic Nucleotide
uguaauggaccucaaacccc

SEQ ID NO: 34 - RNA, 41 nt, Artificial Sequence
  Synthetic Nucleotide
uaauuucuacuaaguguagauaaaaauuacagaagagguug

SEQ ID NO: 35 - DNA, 456 bp, Artificial Sequence
  Synthetic Nucleotide
aagtccaggaaattcattcaccaaatccttttgcgatgcaagctttatggtgataaagcgctcgccgaagggaatggatactgagggaatagcaaggttcacgttctcgttaaaccaaaagcgcagcttaatccagagcgcaagagggggctgatagtatttaggggtttgaggtccattacagctgtaatgaacattacgtcttatgtccagatacgttgcgtccgtgataggagtgatatcttgtttacctgctgtttggatattgtgagagttctcgggaaaatgttgtgaaagaaatttcgggttggtatggctgcacgttcgctgcgtatcattttcatcggtaagaataggtttgctttggtgcggcttgtgcaaatcatgaatgttgcataggagagggccactggttccctccaccgatacctcctggccgaccaagtgcttatatcc

SEQ ID NO: 36 - DNA, 32 bp, Artificial Sequence
  Synthetic Nucleotide
gccgaagggaatggatactgagggaatagcaa

SEQ ID NO: 37 - DNA, 32 bp, Artificial Sequence
  Synthetic Nucleotide
tcccgagaactctcacaatatccaaacagcag

SEQ ID NO: 38 - RNA, 41 nt, Artificial Sequence
  Synthetic Nucleotide
uaauuucuacuaaguguagauggugauuucauacaaaccac

SEQ ID NO: 39 - DNA, 30 bp, Artificial Sequence
  Synthetic Nucleotide
tgatgccatgcgaaatgctggtattgttgg

SEQ ID NO: 40 - DNA, 30 bp, Artificial Sequence
  Synthetic Nucleotide
ctgcagttaaagccctggtcaaggttaata

SEQ ID NO: 41 - RNA, 41 nt, Artificial Sequence
  Synthetic Nucleotide
uaauuucuacuaaguguagaucccccagcgcuucagcguuc

SEQ ID NO: 42 - DNA, 31 bp, Artificial Sequence
  Synthetic Nucleotide
agacgtggtccagaacaaacccaaggaaatt

SEQ ID NO: 43 - DNA, 30 bp, Artificial Sequence
  Synthetic Nucleotide
tgtgtaggtcaaccacgttcccgaaggtgt

SEQ ID NO: 44 - RNA, 51 nt, Artificial Sequence
  Synthetic Nucleotide
gaccaccccaaaaaugaaggggacuaaaaccaacaucagucugauaagcua

SEQ ID NO: 45 - DNA, 11 bp, Artificial Sequence
  Synthetic Nucleotide
tagcttatcag

SEQ ID NO: 46 - DNA, 16 bp, Artificial Sequence
  Synthetic Nucleotide
tagcttatcagactga

SEQ ID NO: 47 - DNA, 21 bp, Artificial Sequence
  Synthetic Nucleotide
tagcttatcagactgatgttg

SEQ ID NO: 48 - DNA, 12 bp, Artificial Sequence
  Synthetic Nucleotide
gttttagtcccc

SEQ ID NO: 49 - DNA, 17 bp, Artificial Sequence
  Synthetic Nucleotide
ccccttcatttttgggg

SEQ ID NO: 50 - DNA, 18 bp, Artificial Sequence
  Synthetic Nucleotide
ttcatttttggggtggtc

SEQ ID NO: 51 - DNA, 21 bp, Artificial Sequence
  Synthetic Nucleotide
gttttagtccccttcattttt

SEQ ID NO: 52 - DNA, 30 bp, Artificial Sequence
  Synthetic Nucleotide
gttttagtccccttcatttttggggtggtc

SEQ ID NO: 53 - DNA, 22 bp, Artificial Sequence
  Synthetic Nucleotide
tagcttatcagactgatgttgg

SEQ ID NO: 54 - DNA, 23 bp, Artificial Sequence
  Synthetic Nucleotide
tagcttatcagactgatgttggt

SEQ ID NO: 55 - DNA, 24 bp, Artificial Sequence
  Synthetic Nucleotide
tagcttatcagactgatgttggtt

SEQ ID NO: 56 - DNA, 25 bp, Artificial Sequence
  Synthetic Nucleotide
tagcttatcagactgatgttggttt

SEQ ID NO: 57 - DNA, 26 bp, Artificial Sequence
  Synthetic Nucleotide
tagcttatcagactgatgttggtttt

SEQ ID NO: 58 - DNA, 25 bp, Artificial Sequence
  Synthetic Nucleotide
agcttatcagactgatgttggtttt

SEQ ID NO: 59 - DNA, 24 bp, Artificial Sequence
  Synthetic Nucleotide
gcttatcagactgatgttggtttt

SEQ ID NO: 60 - DNA, 23 bp, Artificial Sequence
  Synthetic Nucleotide
cttatcagactgatgttggtttt

SEQ ID NO: 61 - DNA, 22 bp, Artificial Sequence
  Synthetic Nucleotide
ttatcagactgatgttggtttt

SEQ ID NO: 62 - DNA, 20 bp, Artificial Sequence
  Synthetic Nucleotide
atcagactgatgttggtttt

SEQ ID NO: 63 - DNA, 25 bp, Artificial Sequence
  Synthetic Nucleotide
atcagactgatgttggttttagtcc

SEQ ID NO: 64 - DNA, 20 bp, Artificial Sequence
  Synthetic Nucleotide
tgatgttggttttagtcccc

SEQ ID NO: 65 - RNA, 22 nt, Artificial Sequence
  Synthetic Nucleotide
uagcuuaucagacugauguuga

SEQ ID NO: 66 - DNA, 931 bp, Artificial Sequence
  Synthetic Nucleotide
ccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtg

SEQ ID NO: 67 - DNA, 931 bp, Artificial Sequence
  Synthetic Nucleotide
ccaaattggctactaccgtagagctaccagacgaattcgtggtggtgacggtaaaatgaaagagctcagccccagatggtatttttactatctaggaactggaccagaagctggacttccctatggtgctaacaaagaaggcatcatatgggttgcaactgagggagccttaaacacaccgaaagaccacattggcacccgcaatcctgctaacaatgctgcaatcgtgctacaacttcctcaaggaacaacattgccaaaaggcttctacgcagaagggagcagaggcggcagtcaagcttcttcacgctcctcatcacgtagtcgcaacagttcaagaaactcaactccaggcagcagtaggggaacttctcctgctagaatggctggcaatggcggtgacactgctcttgctttgctgctgctagataggttgaaccagcttgagaacaaagtatctggcaaaggccaacaacaacagggccaaactgtcactaagaaatctgctgctgaggcatctaaaaagcctcgccaaaaacgtactgctacaaaacagtacaacgtcactcaagcatttgggagacgtggtccagaacaaacccaaggaaattttggggaccaagaattaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagataacgtcatactgctgaataagcacattgacgcatacaaaacattcccaccaacagagcctaaaaaggacaaaaagaaaaaggctgatgaacttcaggctttaccgcagagacagaagaaacaacaaactgtg

(19) United States
(12) Patent Application Publication
(10) Pub. No.: US 2024/0409986 A1
(43) Pub. Date: Dec. 12, 2024

(54) NUCLEIC ACID DETECTION KIT AND DETECTION METHOD BASED ON THE PHOTO-CONTROLLED CRISPR-CAS SYSTEM

(71) Applicant: South China Normal University, Guangzhou City (CN)

(72) Inventors: Xiaoming ZHOU, Guangzhou City (CN); Menglu HU, Guangzhou City (CN)

(21) Appl. No.: 18/268,964

(22) PCT Filed: May 6, 2022
(86) PCT No.: PCT/CN2022/091068

§ 371 Date: Jun. 21, 2023

(30) Foreign Application Priority Data
Jan. 24, 2022 (CN) 202210078298.2

(51) Int. Cl. C12Q 1/6844; C12N 9/22; C12N 15/11

(52) U.S. Cl. CPC C12Q 1/6844; C12N 9/22; C12N 15/11; C12N 2310/20; C12N 2320/10

(57) ABSTRACT

The present invention discloses a nucleic acid detection kit and a detection method based on photo-controlled CRISPR-Cas, wherein the kit comprises silent guide RNA and Cas protein; the silent guide RNA is formed by annealing hybridization of silent nucleotide and guide RNA; the guide RNA, designed according to a target nucleic acid sequence, includes two regions, i.e. a repetitive region and a spacer region; the silent nucleotide is completely complementarily paired with a the of the guide RNA, or is completely paired with a the of the guide RNA; the bases of the silent nucleotide are linked by PC linker; and the Cas protein is Cas12 protein or Cas13 protein. Although this method separates the nucleic acid amplification from the CRISPR-Cas detection in time, it can allow them to be completed in the same closed reaction tube, thereby avoiding the transfer process of uncapped reagent, ensuring that the detection is not affected by aerosol pollution while ensuring high detection sensitivity.

### FIELD OF THE INVENTION

The present invention belongs to the field of biodetection, and specifically relates to a nucleic acid detection kit and a detection method based on the photo-controlled CRISPR-Cas system.

### BACKGROUND OF THE INVENTION

At present, CRISPR-Cas systems, such as CRISPR-Cas12 and CRISPR-Cas13 systems, have been widely used in the field of nucleic acid diagnosis. However, the sensitivity of the CRISPR-Cas detection system is still difficult to reach the level of PCR, so most of the nucleic acid detection methods developed based on CRISPR-Cas need to be combined with nucleic acid amplification.

In a detection method that combines the nucleic acid amplification system and the CRISPR-Cas detection system into a single reaction tube, the CRISPR-Cas detection system will recognize and cleave the target nucleic acid, which will lead to the breakage of amplification templates, thus reducing the amplification efficiency. Besides, in the process of amplification, the initial amplification products will also be recognized and cleaved by the CRISPR-Cas detection system, thus resulting in the breakage of templates that can be recycled, leading to a decline in the amplification efficiency.

These factors lead to the low efficiency of the existing single-tube nucleic acid detection technology based on CRISPR-Cas, which cannot meet the requirements of highly sensitive nucleic acid detection.

Because of these reasons, many reported nucleic acid detection technologies based on CRISPR-Cas carry out the nucleic acid amplification process and CRISPR-Cas detection process step by step. However, the step-by-step process will involve the uncapping of reaction tubes and liquid transfer, which will easily cause aerosol pollution, leading to false positives. Therefore, it is an important scientific topic in this field to develop a single-tube detection method that can improve the sensitivity of CRISPR-Cas system.

### CONTENTS OF THE INVENTION

A purpose of the present invention is to provide a nucleic acid detection kit and a detection method based on the photo-controlled CRISPR-Cas, which can separate the nucleic acid amplification from the CRISPR-Cas detection in time, and can also allow them to be completed in the same closed test tube, thereby avoiding the transfer process of uncapped reagent, ensuring that the detection is not affected by aerosol pollution while ensuring high detection sensitivity.

The purpose of the present invention is achieved through the following technical solution:

A nucleic acid detection kit based on photo-controlled CRISPR-Cas, comprises a silent guide RNA and a Cas protein;
the kit further includes at least one of a fluorescence reporter probe, an amplification primer, an enzyme, and a buffer;
the silent guide RNA is formed by annealing hybridization of a silent nucleotide and a guide RNA;
the guide RNA, designed according to the target nucleic acid sequence, includes two regions, i.e. repetitive region (repetitive sequence) and spacer region (spacer sequence); and the guide RNA is combined with the Cas protein to form the protein-nucleic acid complex, which is guided to recognize and cleave the target nucleic acid;
bases of the silent nucleotide are linked by PC linker that can be photo-activated and photo-degraded; and
the silent nucleotide is completely complementarily paired with the sequence of the of the guide RNA, or is completely paired with the sequence of the region of the guide RNA.

Preferably, in addition to being completely complementarily paired with the sequence of the of the guide RNA, the silent nucleotide is also additionally paired with the by 1-21 base(s), more preferably 5 bases;
the silent nucleotide contains 1-6 PC linker(s), preferably 3 PC linkers;
in the silent nucleotide, each PC linker is inserted at intervals of 5-10 bases, preferably every 6 bases;
the molar concentration ratio of the silent nucleotide to the guide RNA is (1-2):1, preferably 2:1;
the base length of the guide RNA is 30-60, preferably 41 or 51;
the base length of the silent nucleotide is 20-40, preferably 25 or 30; and
the Cas protein is a Cas12 protein or a Cas13 protein, wherein the Cas12 protein and the Cas13 protein have both cis-cleavage activity and trans-cleavage activity, different from a Cas9 protein (only having the cis-cleavage activity); besides, the Cas12 protein is an endonuclease targeting DNA (dsDNA or ssDNA), and can cleave ssDNA based on its activated trans-cleavage activity; and the Cas13 protein is an endonuclease targeting RNA, and can cleave ssRNA based on its activated trans-cleavage activity.

The isothermal amplification primers are designed according to the target nucleic acid sequence; and
the isothermal amplification specifically refers to recombinase polymerase amplification (RPA), loop-mediated isothermal amplification (LAMP), helicase-dependent amplification (HDA), strand displacement amplification (SDA), rolling circle amplification (RCA), isothermal exponential amplification reaction (EXPAR), nuclear acid sequence-based amplification (NASBA), single primer isothermal amplification (SPIA), isothermal and chimeric primer-initiated amplification of nucleic acids (ICAN), and strand exchange amplification (SEA), etc., preferably RPA.

A nucleic acid detection method based on photo-controlled CRISPR-Cas using the above kit, comprises the following steps:
(1) mixing the silent guide RNA, Cas protein, fluorescence reporter probe, amplification primer, enzyme and buffer to prepare a premixed reaction solution, and then adding an analyte containing the target nucleic acid, thus forming a mixed system in a single tube; and
(2) performing isothermal amplification on the above mixed system, and when the isothermal amplification is completed, starting UV irradiation, thus breaking the PC-linker and detaching the silent nucleotide from the guide RNA, then starting the CRISPR-Cas nucleic acid cleavage reaction, and finally detecting the fluorescence signal of the mixed system.

Further, in the mixed system in step (1), the final concentration of the Cas protein can be 10-500 nM, and the final concentration of the silent nucleotide-guide RNA complex can be 10-500 nM, so as to ensure that the cleavage reaction of the Cas protein and the target nucleic acid can occur efficiently, wherein the silent nucleotide-guide RNA complex combines with the Cas protein to form a protein-nucleic acid complex, with the concentration of the silent nucleotide-guide RNA complex corresponding to the concentration of the Cas protein.

Further, in the mixed system in step (1), the final concentration of the fluorescence reporter probe is 200 nM-1 μM, so as to ensure that there are enough fluorescence reporter probes to be cleaved near the target nucleic acid.

Further, reaction temperature of the isothermal amplification in step (2) is 25-65° C., so as to ensure normal amplification reaction of the nucleic acid and non-inactivation of the Cas protein.

Further, reaction time of the isothermal amplification in step (2) is at least 5 min, time of the UV irradiation is at least 5 s, and time of the CRISPR-Cas nucleic acid detection is at least 5 min.

The principle of the present invention is as follows:

With the Cas protein combined with the guide RNA to form a protein-nucleic acid complex, when there is a target nucleic acid, the protein-nucleic acid complex, mediated by the guide RNA, will recognize and cleave the target nucleic acid, and meanwhile the trans-cleavage activity of Cas protein will be activated so that the Cas protein will cleave the surrounding fluorescence reporter probe in a non-specific way; and the fluorescence reporter probe is connected with fluorescein at one end and a quencher at the other end, and can be activated to generate a fluorescence signal after being cleaved.

In the present invention, it is designed that the silent nucleotide is complementary with the guide RNA, thereby blocking the recognition of the target nucleic acid by the guide RNA; besides, it is designed that the silent nucleotide is connected with a photo-activated linker, and can be easily broken by UV activation, thereby reviving the CRISPR-Cas detection system.

When this inactivated CRISPR-Cas detection system is combined with the nucleic acid amplification technology in one-tube reaction, the nucleic acid amplification template will not be cleaved by CRISPR-Cas, so the amplification efficiency will not be affected. When the nucleic acid amplification is completed, the CRISPR-Cas detection system is photo-activated.

Although this detection method separates the nucleic acid amplification from the CRISPR-Cas detection in time, it can allow them to be completed in the same closed test tube, thereby avoiding the transfer process of uncapped reagent, ensuring that the detection is not affected by aerosol pollution while ensuring high detection sensitivity.

The present invention has the following advantages and effects with respect to the prior art:
1. Compared with the step-by-step reaction system, the kit and method of the present invention avoid the generation of aerosol pollution and simplify the experimental steps;
2. compared with the one-tube reaction system based on phase separation, the kit and method of the present invention simplify the reaction system and experimental steps;
3. compared with the one-tube reaction system where the Cas reaction and the amplification reaction occur at different positions in the tube, the kit and method of the present invention solve the problem of instability, and do not need to rely on centrifugal equipment; and
4. compared with an optimized Cas reaction system (requiring, for example, two or more guide RNAs), the kit and method of the present invention can achieve highly sensitive nucleic acid detection by using only one guide RNA, simplifying the reaction system and experimental steps.

### BRIEF DESCRIPTION OF THE DRAWINGS

FIG. 1 shows the fluorescence signal intensity caused by different silent DNAs;

FIG. 2 shows the fluorescence signal intensity caused by silent guide RNAs;

FIG. 3 shows the fluorescence signal intensity caused by different silent RNAs;

FIG. 4 shows the fluorescence intensity curve when the kit and method of the present invention are used to detect targets;

FIG. 5 shows the fluorescence intensity curve when the kit and method of the present invention are used to detect targets;

FIG. 6 shows the fluorescence intensity when the kit and method of the present invention are used to detect different targets;

FIG. 7 shows the fluorescence intensity curve when the kit and method of the present invention are used to detect different amounts of targets;

FIG. 8 shows the fluorescence intensity curve when the traditional CRISPR-Cas12 method is used to detect targets;

FIG. 9 shows the electrophoretic diagram when the agarose gel electrophoresis is used to detect targets;

FIG. 10 shows the fluorescence signal intensity caused by different silent DNAs; and

FIG. 11 shows the fluorescence signal intensity caused by silent guide RNAs.

### DETAILED DESCRIPTION OF THE EMBODIMENTS

The present invention will be described in further detail below with reference to examples and drawings, but the embodiments of the present invention are not limited thereto.

### Example 1

### Screening of Silent DNA (Unmodified PC Linker)

### (1) Preparation of Silent Guide RNA

The guide RNA was inactivated by annealing and hybridization methods. The annealing premixed reaction solution contained guide RNA, silent DNA, and Cas protein reaction buffer (NEBuffer 2.1, purchased from New England Biotechnology (Beijing) Co., Ltd., the same below). The final concentration of each component in the annealing premixed reaction solution was 10 μM for the guide RNA, 20 μM for the silent DNA, and 1× for the Cas protein reaction buffer, respectively. The premixed reaction solution was placed on the nucleic acid amplification instrument, then incubated at 70° C. for 5 min, and then gradually cooled down to room temperature, thus obtaining the silent guide RNA.

Guide RNA: uaauuucuacuaaguguagaugggguuugagguccauuaca (SEQ. ID. NO.1), wherein the base in italics was the, and the rest was the.

The code, sequence and serial number of the silent DNA were shown in Table 1.

TABLE 1

| Code | Sequence | Serial number |
|---|---|---|
| S5R20 | accccatctacacttagtagaaatt | SEQ. ID. NO. 2 |
| S5R19 | accccatctacacttagtagaaat | SEQ. ID. NO. 3 |
| S5R18 | accccatctacacttagtagaaa | SEQ. ID. NO. 4 |
| S5R17 | accccatctacacttagtagaa | SEQ. ID. NO. 5 |
| S5R16 | accccatctacacttagtaga | SEQ. ID. NO. 6 |
| S5R14 | accccatctacacttagta | SEQ. ID. NO. 7 |
| S5R13 | accccatctacacttagt | SEQ. ID. NO. 8 |
| S5R12 | accccatctacacttag | SEQ. ID. NO. 9 |
| S5R11 | accccatctacactta | SEQ. ID. NO. 10 |
| S5R10 | accccatctacactt | SEQ. ID. NO. 11 |
| S10R15 | ctcaaaccccatctacacttagtag | SEQ. ID. NO. 12 |
| S9R15 | tcaaaccccatctacacttagtag | SEQ. ID. NO. 13 |
| S8R15 | caaaccccatctacacttagtag | SEQ. ID. NO. 14 |
| S7R15 | aaaccccatctacacttagtag | SEQ. ID. NO. 15 |
| S6R15 | aaccccatctacacttagtag | SEQ. ID. NO. 16 |
| S5R15 | accccatctacacttagtag | SEQ. ID. NO. 17 |
| S4R15 | ccccatctacacttagtag | SEQ. ID. NO. 18 |
| S3R15 | cccatctacacttagtag | SEQ. ID. NO. 19 |
| S2R15 | ccatctacacttagtag | SEQ. ID. NO. 20 |
| S1R15 | catctacacttagtag | SEQ. ID. NO. 21 |
| R15 | atctacacttagtag | SEQ. ID. NO. 22 |
| R21 | atctacacttagtagaaatta | SEQ. ID. NO. 23 |
| S-17 | tgtaatggacctcaaac | SEQ. ID. NO. 24 |
| S-16 | tgtaatggacctcaaa | SEQ. ID. NO. 25 |
| S-15 | tgtaatggacctcaa | SEQ. ID. NO. 26 |
| S-14 | tgtaatggacctca | SEQ. ID. NO. 27 |
| S-13 | tgtaatggacctc | SEQ. ID. NO. 28 |
| S-12 | tgtaatggacct | SEQ. ID. NO. 29 |
| S-11 | tgtaatggacc | SEQ. ID. NO. 30 |

As for the meaning of the code of silent DNA, with the sequence of guide RNA divided into two pants, repetitive region (repetitive sequence) and spacer region (spacer sequence), the complementary pairing between the silent DNA and the guide RNA corresponding to the repetitive region is called R for short, and that corresponding to the spacer region is called S 0.7 for short, different numbers representing the length of bases complementarily paired.

### (2) Preparation of Target DNA

The target used was a double-stranded DNA formed by annealing, and the sequence of the target is as follows:

Target F:
(SEQ. ID. NO. 31)
ctgatagtatttaggggtttgaggtccattacagctgtaatgaacattac
gtcttatgt
Target R:
(SEQ. ID. NO. 32)
acataagacgtaatgttcattacagctgtaatggacctcaaacccctaaa
tactatcag

The annealing premixed reaction solution contained the target F, the target R, and the Cas protein reaction buffer. The final concentration of each component in the annealing premixed reaction solution was 10 μM for the target F, 10 μM for the target R, and 1× for the Cas protein reaction buffer, respectively. The premixed reaction solution was placed on a nucleic acid amplification instrument, then incubated at 70° C. for 5 min, and then gradually cooled down to room temperature, thus obtaining the target DNA.

### (3) Preparation of Cas Protein Premixed Reaction Solution

The Cas protein premixed reaction solution contained a Cas protein reaction buffer, a Cas12 protein (purchased from Guangzhou Bio-lifesci Biotechnology Co., Ltd., the same below), a silent guide RNA, an FQ probe, and a target DNA. The final concentration of each component in the reaction solution was 1× for the Cas protein reaction buffer, 100 nM for the Cas12 protein, 10 nM for the silent guide RNA, 400 nM for the FQ probe, and 1 nM for the target, respectively. The FQ probe used in this example was FQC6 (purchased from Huzhou Hippo Biotechnology Co., Ltd.), with the sequence of FAM-CCCCCC-BHQ1.

The Cas protein premixed reaction solution was incubated at 37° C. for 30 min. The fluorescence signal intensity of the Cas protein premixed reaction solution was determined every minute.

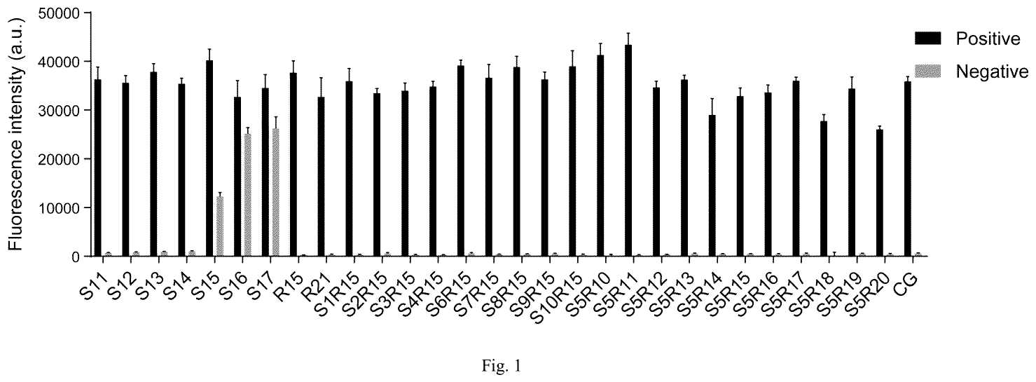

In FIG. 1, the target DNA was added to the positive group (P), RNase-free water was used to replace the target DNA in the negative group (N), and the CG group contained no silent DNA.

The experimental results were shown in FIG. 1, indicating that the silent DNAs which were complementarily paired with the guide RNA at different positions to block their activity could not effectively silence the activity of the guide RNA.

However, in FIG. 1, when the three silent DNAs S15, S16 and S17 were used to block the activity of guide RNA, even the negative control group without the target DNA could generate fluorescence signals, which might be because these three silent DNAs were enough to be the target, to activate the cleavage activity of Cas protein.

### Example 2

Verification of blocking effect of silent RNA (without PC linker modification, completely complementarily paired with the sequence of the spacer region of guide RNA)

### (1) Preparation of Silent Guide RNA

The guide RNA is inactivated by annealing and hybridization methods. The annealing premixed reaction solution contained guide RNA, silent RNA, and Cas protein reaction buffer (NEBuffer 2.1, purchased from New England Biotechnology (Beijing) Co., Ltd., the same below). The final concentration of each component in the annealing premixed reaction solution was 10 μM for the guide RNA, 20 μM for the silent RNA, and 1× for the Cas protein reaction buffer, respectively. The premixed reaction solution was placed on a nucleic acid amplification instrument, then incubated at 70° C. for 5 min, and then gradually cooled down to room temperature, thus obtaining the silent guide RNA.

The sequence of silent RNA was uguaauggaccucaaacccc (SEQ. ID. No.33), and the sequence of guide RNA (i.e. “paired crRNA” in Table 2) was SEQ ID. NO.1.

(2) Preparation of the target DNA is the same as in Example 1.

(3) Preparation of Cas protein premixed reaction solution is the same as in Example 1.

(4) The Cas protein premixed reaction solution was incubated at 37° C. for 60 min. The fluorescence signal intensity of the Cas protein premixed reaction solution was determined every minute.

The main sample formulas of individual experimental groups in FIG. 2 were shown in Table 2. The “mismatched crRNA” in Table 2 was the guide RNA that was incompletely complementarily paired with the silent RNA (SEQ. ID. NO.33), and its sequence was uaauuucuacuaaguguagauaaaaauuacagaagagguug (SEQ. ID. NO.34). For the experimental group without the target, RNase-free water was used instead.

TABLE 2

| | Paired | Mismatched | Silent | Cas12a | |
|---|---|---|---|---|---|
| | crRNA | crRNA | RNA | protein | Target |
| 1 | + | | | + | + |
| 2 | + | | | + | |
| 3 | + | | + | + | + |
| 4 | + | | + | + | |
| 5 | | + | | + | + |
| 6 | | + | | + | |
| 7 | | + | + | + | + |
| 8 | | + | + | + | |

The results were shown in FIG. 2, indicating that the silent RNA completely complementarily paired with the sequence of the spacer region of the guide RNA achieved an excellent silencing effect, with almost no fluorescence signal generated (Experiment 3); however, the guide RNA that was not completely complementarily paired with the silent RNA still had a strong fluorescence signal after being blocked (Experiment 7).

These results indicated that the silent RNA that was completely complementarily paired with the spacer region of the guide RNA could be used to completely block the activity of the guide RNA; moreover, the addition of the silent RNA to the Cas12a cleavage system that was mismatched with the guide RNA would not affect this system.

### Example 3

Investigation of activity blocking effect of silent RNA (different amounts of PC linkers, different blocking positions, different dosage ratios) on guide RNA, and activity recovery effect of guide RNA after being irradiated

(1) The guide RNA was inactivated by hybridization, and the silent RNA (SEQ. ID. NO.33) and the guide RNA (SEQ. ID. NO.1) were added according to a molar concentration ratio of 1:1, 1.5:1 and 2:1, respectively. The 10 μM guide RNA, silent RNA, 1×Cas protein reaction buffer were mixed to prepare a premixed reaction solution. The premixed reaction solution was placed on a nucleic acid amplification instrument, then allowed to react at 70° C. for 5 min, and then gradually cooled down to room temperature, thus obtaining the silent guide RNA.

The sequences modified with PC-linker, which were used in the present invention, was purchased from Huzhou Hippo Biotechnology Co., Ltd.

(2) Preparation of the target DNA is the same as in Example 1.

(3) Preparation of Cas protein premixed reaction solution is the same as in Example 1.

(4) The Cas protein premixed reaction solution was incubated at 37° C. for 60 min. The fluorescence signal intensity of the Cas protein premixed reaction solution was determined every minute.

The sequence of silent RNAs 6PC, 3PC and 2PC used in this example was the same as that of SEQ ID. NO.33, with different numbers of PC linkers inserted in the bases thereof. In addition to being completely complementarily paired with the sequence of spacer region of the guide RNA, R5-3PC was also additionally paired with the by 5 bases.

Specifically, the code and sequence of silent RNAs were shown in Table 3:

TABLE 3

| Code | Sequence |
|---|---|
| 6PC | ugu(PC-linker)aau(PC-linker)gga(PC-linker)ccu |
| | (PC-linker)--caa(PC-linker)acc(PC-linker)cc |
| 3PC | uguaa(PC-linker)uggac(PC-linker)cucaa |
| | (PC-linker)acccc |
| 2PC | uguaaug(PC-linker)gaccuca(PC-linker)aacccc |
| R5-3PC | uguaau(PC-linker)ggaccu(PC-linker)caaacc |
| | (PC-linker)ccaucua |

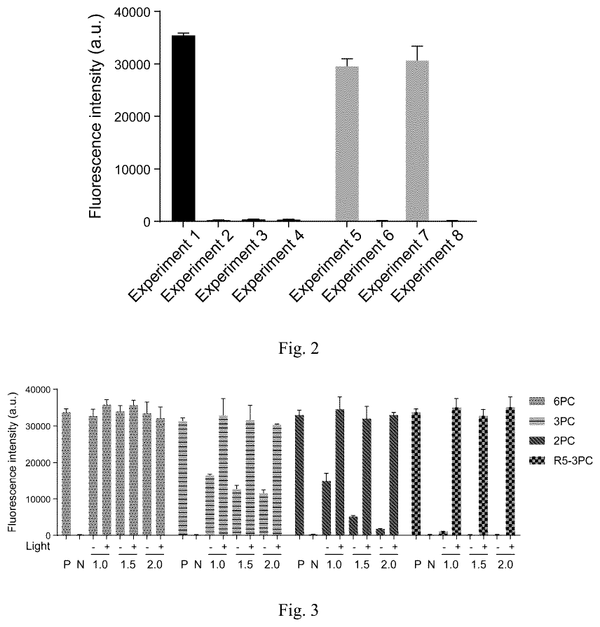

In FIG. 3, + represented that the premixed reaction solution was UV-irradiated for 30 s before step (4), and − represented that no irradiation treatment was performed before step (4).

The results were shown in FIG. 3, indicating that the best effect could be achieved when the silent guide RNA of R5-3PC was used.

It could be seen from the result that whether the molar concentration ratio of the silent RNA to the guide RNA used was 1:1, 1.5:1 or 2:1, the activity of guide RNA after being irradiated was equivalent to that of the positive control group; however, the blocking effect could be seen from the no-irradiation group, that is, the lower the signal value, the better the blocking effect; it could be seen from the figure that the best blocking effect was achieved when the molar concentration ratio of the silent RNA to the guide RNA was 2:1 and the blocking RNA used was R5-3PC.

### Example 4

Verification of feasibility of photo-controlled CRISPR-Cas12 detection system (the target DNA was a plasmid DNA (SEQ. ID. NO.35) containing partial sequence of the B646L gene of African swine fever virus (ASFV))

(1) A guide RNA (SEQ. ID. NO.1) was designed according to the B646L gene of ASFV. The silent RNA sequence used was R5-3PC.

(2) Preparation of the silent guide RNA is the same as in Example 2.

(3) A Cas protein premixed reaction solution was prepared, containing Cas protein reaction buffer, Cas12 protein, silent guide RNA, and FQ probe. The final concentration of each component in the reaction solution was 1× for the Cas protein reaction buffer, 100 nM for the Cas protein, 100 nM for the silent guide RNA, and 400 nM for the FQ probe, respectively. The FQ probe used in this example was FQC6, with the sequence of FAM-CCCCCC-BHQ1.

(4) Preparation of amplification reaction solution: In this example, the RPA method was used to amplify the DNA target, using the TwistAmp Basic Kit (article No. 111781). The RPA reaction solution contained, primers, amplification reaction buffer, amplification enzymes, plasmid DNA (synthesized by Nanjing Tsingke Biotechnology Co., Ltd.) containing partial sequence of the B646L gene of ASFV, and magnesium acetate. The final concentration of each component in the reaction solution was 480 nM for the primers, 1× for the amplification reaction buffer, 100 ag/NL for the target, and 14 mM for the magnesium acetate, respectively.

Forward primer sequence:
(SEQ. ID. NO. 36)
gccgaagggaatggatactgagggaatagcaa;
and
Reverse primer sequence:
(SEQ. ID. NO. 37)
tcccgagaactctcacaatatccaaacagcag.

(5) The Cas protein reaction solution and the amplification reaction solution were mixed to prepare a premixed reaction solution, which was then incubated at 37° C. After 30 min, the premixed reaction solution was taken out and then UV-irradiated for 30 s. The fluorescence signal intensity of premixed reaction solution was measured by a real-time quantitative PCR instrument.

In this example, the target DNA was added to the positive group (P), while RNase-free water was used to replace the target DNA in the negative group (N).

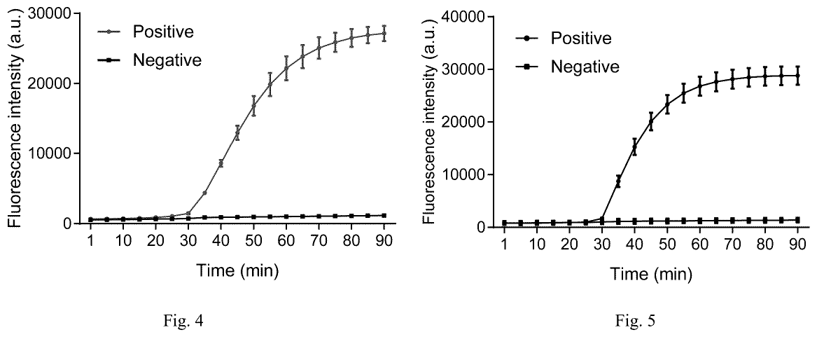

The experimental results were shown in FIG. 4. The positive group and negative group exhibit great signal differences, indicating that the positive group had a good signal-to-noise ratio and reaction rate.

### Example 5

Verification of feasibility of the photo-controlled CRISPR-Cas12 detection system (the target RNA was the O gene of Severe Acute Respiratory Syndrome Coronavirus 2 (SARS-CoV-2) (genome location 13201-15600, GenBank No. NC_045512))

(1) For the O gene of SARS-CoV-2, the sequence of guide RNA was designed to be uaauuucuacuaaguguagauggugauuucauacaaaccac (SEQ. ID. NO.38), wherein the base in italics was the repetitive region, and the rest was the spacer region. The silent RNA sequence used was gugguu(PC-linker)uguaug(PC-linker)aaauca(PC-linker)ccaucua.

(2) Preparation of the silent guide RNA is the same as in Example 4.

(3) Preparation of the Cas protein premixed reaction solution is the same as in Example 4.

(4) Preparation of the reverse transcription amplification reaction solution.

A reverse transcription recombinase polymerase amplification (RT-RPA) method was used to amplify the RNA target, using the TwistAmp Basic Kit (article No. 111781). The RT-RPA reaction solution contained primers, amplification buffer, amplification enzymes, reverse transcriptase, target, and magnesium acetate. The final concentration of each component in the reaction solution was 480 nM for the primers, 1× for the amplification buffer, 5 U/μL for the reverse transcriptase, 100 aM for the target, and 14 mM for the magnesium acetate, respectively.

(5) Same as in Example 4.

Forward primer sequence:
(SEQ. ID. NO. 39)
tgatgccatgcgaaatgctggtattgttgg;
and
Reverse primer sequence:
(SEQ. ID. NO. 40)
ctgcagttaaagccctggtcaaggttaata.

The target used in this example was an RNA reference material purchased from the National Institute of Metrology, China and containing part of the O gene sequence of SARS-CoV-2 (genome location 13201-15600, GenBank No. NC_045512) (the same below).

In this example, the target RNA was added to the positive group (P), while RNase-free water was used to replace the target RNA in the negative group (N).

The results were shown in FIG. 5, showing that when the method was applied to the detection of SARS-CoV-2, the signal difference between the positive group and the negative group was very large, indicating that the positive group had a good signal-to-noise ratio and reaction rate.

### Example 6

Specific detection based on N gene of SARS-CoV-2.

(1) For the N gene of SARS-CoV-2 (genome location 28274-29533, GenBank No. MN908947.3), the sequence of guide RNA was designed to be uaauuucuacuaaguguagaucccccagcgcuucagcguuc (SEQ. ID. NO.41), wherein the base in italics was the repetitive region, and the rest was the spacer region. The silent RNA sequence used was gaacgc(PC-linker)ugaagc(PC-linker)gcuggg(PC-linker)ggaucua.

The remaining steps were the same as those in Example 5.

Forward primer sequence:
(SEQ. ID. NO. 42)
agacgtggtccagaacaaacccaaggaaatt;
and
Reverse primer sequence:
(SEQ. ID. NO. 43)
tgtgtaggtcaaccacgttcccgaaggtgt.

The added targets were as follows: an RNA standard of SARS-CoV-2 (purchased from the Chinese Academy of Metrology), a SARS-CoV RNA (SEQ. ID. NO.66), and a bat-SL-CoVZC45 RNA (SEQ. ID. NO.67).

The RNA sequence of SARS-CoV and bat-SL-CoVZC45 were homemade by in vitro transcription of DNA plasmid, and the DNA plasmid were synthesized by biological company.

RNase-free water was added as the target in the NTC group.

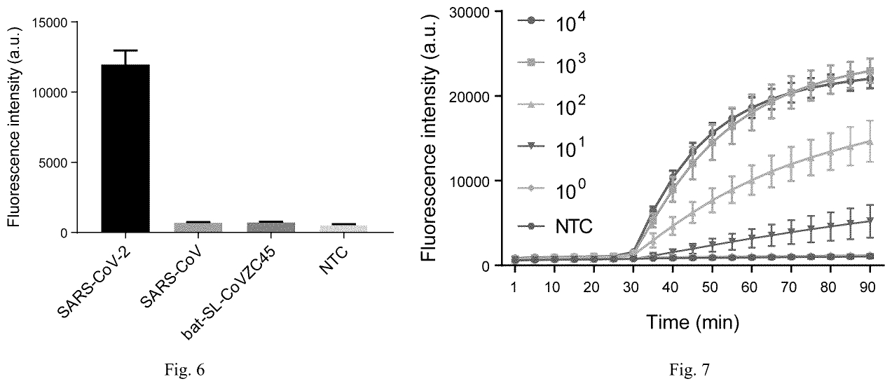

The results were shown in FIG. 6, indicating that this method had good detection specificity when applied to the specificity detection of SARS-CoV-2. In addition, it is worth pointing out that this method is expected to be applicable to the detection of single-base mutation.

### Example 7

### Sensitivity Detection Based on O Gene of SARS-CoV-2

The experimental materials and steps were the same as those in Example 5.

The amounts of targets added to the reaction solution were 104 copies, 103 copies, 10² copies, 10¹ copies, 10⁰ copies, and 0 copies (NTC).

The results were shown in FIG. 7, indicating that when applied to the sensitivity detection of SARS-CoV-2, this method had good detection sensitivity, with the minimum detection limit reaching 10 copies.

### Comparative Example 1

In this Comparative Example 1 was a conventional one-pot DNA detection method based on CRISPR-Cas12, where the target DNA, Cas12a protein, guide RNA, fluorescence reporter probe and buffer solution of Example 1 were used.

(1) The sequence of guide RNA (SEQ. ID. NO.1) was designed according to the B646L gene of ASFV.

(2) Cas protein premixed reaction solution was prepared, containing Cas protein reaction buffer, Cas protein, guide RNA, and FQ probe. The final concentration of each component in the reaction solution was 1× for the Cas protein reaction buffer, 100 nM for the Cas protein, 100 nM for the guide RNA, and 400 nM for the FQ probe, respectively. The FQ probe used in this example was FQC6, with the sequence of FAM-CCCCCC-BHQ1.

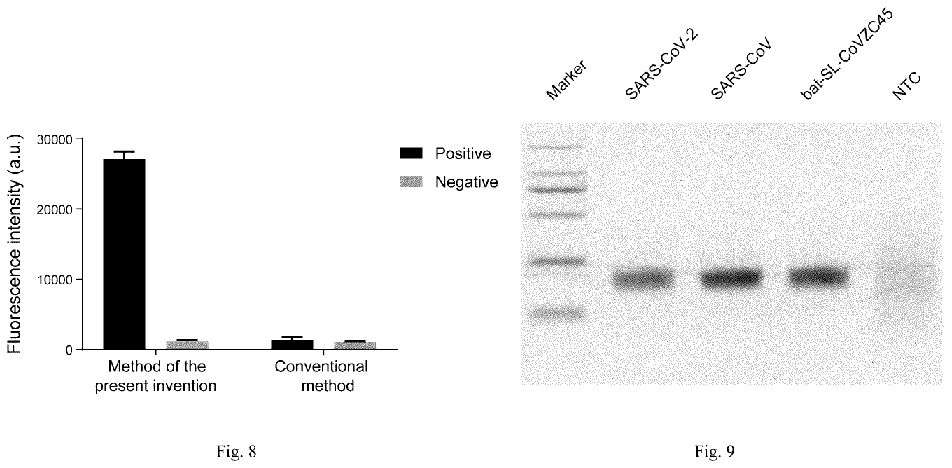

In FIG. 8, the “method of the present invention” represents the addition of the silent guide RNA to the Cas protein premixed reaction solution, with the preparation method being the same as that in Example 4 and the silent RNA sequence being R5-3PC.

The remaining steps of the conventional one-pot DNA detection method were the same as those in Example 4, except that no irradiation treatment was performed.

In this example, the target DNA was added to the positive group (P), while RNase-free water was used to replace the target DNA in the negative group (N).

As shown in FIG. 8, a comparison of the detection efficiency of the conventional one-pot DNA detection method and the method of the prevent invention for the detection of the same concentration targets (100 ag/μL) was carried out. The method of the present invention had a very strong fluorescence signal intensity (FIG. 8), while the conventional CRISPR-Cas12 assay has almost no fluorescence intensity (FIG. 8), indicating that the proposed method of the present invention could significantly improve the sensitivity of the conventional one-pot detection method.

### Comparative Example 2

In this Comparative Example 2, agarose gel electrophoresis was used to verify the specificity of RT-RPA amplification of SARS-CoV-2 RNA standard, SARS-CoV RNA, and bat-SL-CoVZC45 RNA. The target RNA and RT-RPA amplification system used in this example are the same as in Example 6.

The RT-RPA amplification reaction solution, being the same as that in Example 6, was incubated at 37° C. for 30 min. Then the agarose gel electrophoresis was used to verify the amplification product.

As shown in FIG. 9, the agarose gel electrophoresis was performed to verify the RT-RPA amplification products of the SARS-CoV-2 RNA, the SARS-CoV RNA, and the bat-SL-CoVZC45 RNA. Amplified bands were observed in the amplification products of all three viral RNAs except for NTC (with the target being replaced by RNase-free water), indicating that only depending on the specificity of RT-RPA amplification was not enough for distinguishing the three viruses.

In example 6, the specificity analysis of these three viruses using the method of the present invention showed no fluorescence signal except for the amplification of the SARS-CoV-2 RNA standard, indicating that the method proposed in this patent has better specificity.

### Example 8

Screening of Silent DNA (without PC Linker) for Cas13 System

(1) Preparation of silent guide RNA: The annealing hybridization method was used to inactivate a guide RNA. An annealing premixed reaction solution contained guide RNA, silent DNA, and Cas protein reaction buffer (PCR buffer, purchased from Takara Biomedical Technology (Beijing) Co., Ltd., the same below). The final concentration of each component in the annealing premixed reaction solution was 10 μM for the guide RNA, 20 μM for the silent DNA, and 1× for the Cas protein reaction buffer, respectively. The premixed reaction solution was placed on a nucleic acid amplification instrument, then incubated at 70° C. for 5 min, and then gradually cooled down to room temperature, thus obtaining a silent guide RNA.

Guide RNA: gaccaccccaaaaaugaaggggacuaaaaccaacaucagucugauaagcua (SEQ. ID. NO. 44), wherein the base in italics was the, and the rest was the.

The code, sequence and serial number of the silent DNA were shown in Table 4.

TABLE 4

| Code | Sequence | Serial number |
|---|---|---|
| S11 | tagcttatcag | SEQ. ID. NO. 45 |
| S16 | tagcttatcagactga | SEQ. ID. NO. 46 |
| S21 | tagcttatcagactgatgttg | SEQ. ID. NO. 47 |
| R12 | gttttagtcccc | SEQ. ID. NO. 48 |
| R17 | ccccttcatttttgggg | SEQ. ID. NO. 49 |
| R18 | ttcatttttggggtggtc | SEQ. ID. NO. 50 |
| R21 | gttttagtccccttcattttt | SEQ. ID. NO. 51 |
| R30 | gttttagtccccttcatttttggggt | SEQ. ID. NO. 52 |
| | ggtc | |
| S21R1 | tagcttatcagactgatgttgg | SEQ. ID. NO. 53 |
| S21R2 | tagcttatcagactgatgttggt | SEQ. ID. NO. 54 |
| S21R3 | tagcttatcagactgatgttggtt | SEQ. ID. NO. 55 |
| S21R4 | tagcttatcagactgatgttggttt | SEQ. ID. NO. 56 |
| S21R5 | tagcttatcagactgatgttggtttt | SEQ. ID. NO. 57 |
| S20R5 | agcttatcagactgatgttggtttt | SEQ. ID. NO. 58 |
| S19R5 | gcttatcagactgatgttggtttt | SEQ. ID. NO. 59 |
| S18R5 | cttatcagactgatgttggtttt | SEQ. ID. NO. 60 |
| S17R5 | ttatcagactgatgttggtttt | SEQ. ID. NO. 61 |
| S15R5 | atcagactgatgttggtttt | SEQ. ID. NO. 62 |
| S15R10 | atcagactgatgttggttttagtcc | SEQ. ID. NO. 63 |
| S8R12 | tgatgttggttttagtcccc | SEQ. ID. NO. 64 |

As for the meaning of the code of silent DNA, with the sequence of guide RNA divided into two pants, repetitive region (repetitive sequence) and spacer region (spacer sequence), the complementary pairing between the silent DNA and the guide RNA corresponding to the repetitive region is called R for short, and that corresponding to the spacer region is called S for short, different numbers representing the length of bases complementarily paired.

(2) A Cas protein premixed reaction solution was prepared, containing Cas protein reaction buffer, Cas13 protein (purchased from Guangzhou Bio-lifesci Biotechnology Co., Ltd., the same below), silent guide RNA, FQ probe, and target RNA (same as Example 1). The final concentration of each component in the reaction solution was 1× for the Cas protein reaction buffer, 100 nM for the Cas13 protein, 10 nM for the silent guide RNA, 400 nM for the FQ probe, and 1 nM for the target, respectively. The FQ probe used in this example was FQU5 (purchased from Huzhou Hippo Biotechnology Co., Ltd.), with the sequence of FAM-UUUUU-BHQ1.

The target used was single-stranded RNA, with the target sequence of uagcuuaucagacugauguuga (SEQ. ID. NO.65).

(3) The Cas protein premixed reaction solution was incubated at 37° C. for 30 min. The fluorescence signal intensity of the Cas protein premixed reaction solution was determined every minute.

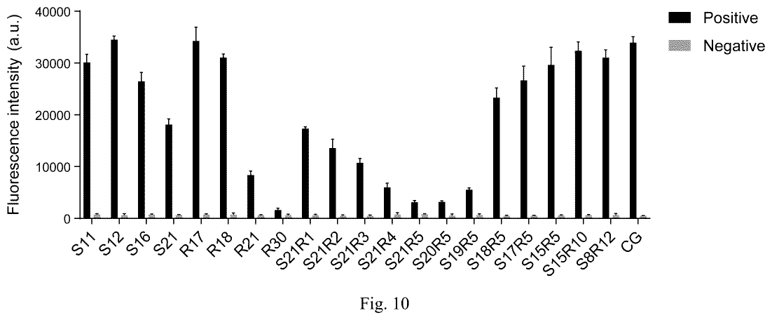

In FIG. 10, the target RNA was added to the positive group (P), RNase-free water was used to replace the target RNA in the negative group (N), and the CG group contained no silent DNA.

The results were shown in FIG. 10, showing that when the R30 sequence (completely paired with the sequence of repetitive region of the guide RNA) was used to silence the activity of the guide RNA, the fluorescence signal intensity was the lowest, indicating that the silencing effect of this sequence was the best.

### Example 9

Investigation the activity blocking effect of silent DNA (with PC linker, different dosage ratios) on guide RNA in Cas13 system, and the activity recovery effect of guide RNA after being irradiated

(1) Preparation of silent guide RNA: The guide RNA was inactivated by hybridization, and the silent DNA and the guide RNA were added according to a molar concentration ratio of 1:1, 1.5:1 and 2:1, respectively. The 10 μM guide RNA (SEQ. ID. NO.44), different concentrations of silent DNA (gttttagt(PC-linker)ccccttc(PC-linker)atttttg(PC-linker)gggtggtc), and 1×Cas protein reaction buffer were mixed to prepare a premixed reaction solution. The premixed reaction solution was placed on a nucleic acid amplification instrument, then allowed to react at 70° C. for 5 min, and then gradually cooled down to room temperature.

Steps (2)-(3) were the same as those in Example 8.

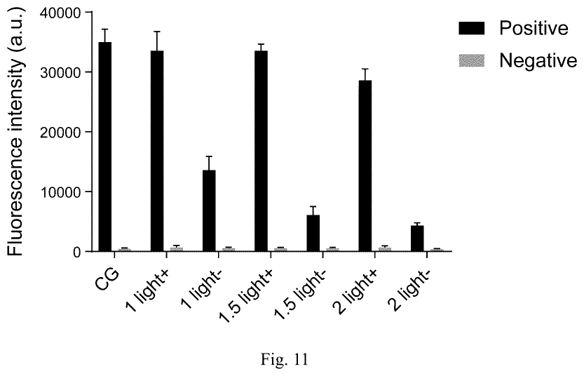

In FIG. 11, the target RNA was added to the positive group (P), RNase-free water was used to replace the target RNA in the negative group (N), and the CG group contained no silent DNA; the fluorescence intensity shown in this figure was the fluorescence signal intensity of the Cas protein premixed reaction solution which had reacted for 60 min; and + represented that the premixed reaction solution was UV-irradiated for 30 s before step 3, and − represented that no irradiation treatment was performed before step 3.

It could be seen from the figure that whether the molar concentration ratio of the silent DNA to the guide RNA used was 1:1, 1.5:1 or 2:1, the activity of guide RNA after being irradiated was equivalent to that of the positive control group; however, the blocking effect could be seen from the no-irradiation group, that is, the lower the signal value, the better the blocking effect; it could be seen from the figure that the best blocking effect was achieved when the molar concentration ratio of the silent DNA to the guide RNA was 2:1.

The above examples are preferred embodiments of the present invention, but the embodiments of the present invention are not limited thereto, and any other alterations, modifications, replacements, combinations, and simplifications made without departing from the spirit and principle of the present invention shall all be equivalent substitutions and included in the scope of protection of the present invention.

1. A photo-controlled CRISPR-Cas nucleic acid detection kit, comprising a silent guide RNA and a Cas protein;
the silent guide RNA is formed by an annealing hybridization of a silent nucleotide and a guide RNA;
the guide RNA, designed according to a target nucleic acid sequence, includes two regions, i.e. a repetitive region and a spacer region;
the silent nucleotide is completely complementarily paired with a the of the guide RNA, or is completely paired with a the of the guide RNA;
bases of the silent nucleotide are linked by a PC linker; and
the Cas protein is Cas12 protein or Cas13 protein.

2. The kit according to claim 1, wherein: in addition to being completely complementarily paired with the sequence of the spacer region of the guide RNA, the silent nucleotide is also additionally paired with the by one or more bases.

3. The kit according to claim 1, wherein: the silent nucleotide contains 1-6 PC linker(s), each of which is inserted at intervals of 5-10 bases.

4. The kit according to claim 1, wherein: a molar concentration ratio of the silent nucleotide to the guide RNA is (1-2):1.

5. The kit according to claim 1, wherein: the silent nucleotide contains 3 PC linkers.

6. The kit according to claim 1, wherein: in the silent nucleotide, each PC linker is inserted at intervals of 6 bases.

7. The kit according to claim 1, wherein: the molar concentration ratio of the silent nucleotide to the guide RNA is 2:1.

8. The kit according to claim 1, wherein: the kit further includes at least one of a fluorescence reporter probe, an amplification primer, an enzyme, and a buffer.

9. A nucleic acid detection method based on photo-controlled CRISPR-Cas wherein: the kit according to claim 1 is used.

10. The detection method according to claim 9, wherein the method comprises the following steps:
(1) mixing a silent guide RNA, a Cas protein, a fluorescence reporter probe, an amplification primer, an enzyme and a buffer to prepare a premixed reaction solution, and then adding an analyte containing the target nucleic acid, thus forming a mixed system in a single tube; and
(2) performing isothermal amplification on the above mixed system, and when the isothermal amplification is completed, starting UV irradiation, thus breaking the PC-linker and detached the silent nucleotide from the guide RNA, then starting a CRISPR-Cas nucleic acid cleavage reaction, and finally detecting a fluorescence signal of the mixed system.
